(19) United States
(12) Patent Application Publication
(10) Pub. No.: US 2009/0043151 A1
(43) Pub. Date: Feb. 12, 2009

(54) Device for closing a natural or artificial anus

(76) Inventor: Fred Gobel, Wilhelmsfeld (DE)

Correspondence Address: Stephen E. Bondura;Dority & Manning, P.A., P.O. Box 1449, Greenville, SC, 29602-1449, US

(21) Appl. No.: 11/658,381

(22) PCT Filed: Jul. 21, 2005
(86) PCT No.: PCT/EP2005/007950

§ 371 Date: Oct. 21, 2008

(30) Foreign Application Priority Data
Jul. 23, 2004 (EP) 2004/008256
Feb. 9, 2005 (DE) 10 2005 005 988.0
Apr. 15, 2005 (DE) 10 2005 017 652.6
May 6, 2005 (DE) 10 2005 021 081.3

(51) Int. Cl. A61F 2/00

(52) U.S. Cl. 600/31; 606/192

(57) ABSTRACT

The invention relates to device (1; 1′) for closing a natural or artificial anus, comprising an inflatable balloon (2) with an approximately toroidal structure made from a flat tube section, which is turned inside out and whose both ends (7, 8) extend coaxially into one another and are each joined to a sleeve (9). The outer layer (5) of the tube section that is turned inside out has a radially enlarged, patient-proximal area (3) for inserting into the rectum and has a patient-distal area (11, 7, 8), which is tapered thereto and which remains, at least in areas, outside of the rectum during use. The tube sections have, in the transanal area (11, 7, 8), a material hardness H₁ of greater than 60 according to Shore hardness test A. According to the invention, a stiffening sleeve (14) is placed inside the interrectal area (3) of the inner layer (5) in such a manner that this sleeve is completely separated from the hollow space (10) inside the balloon (2) by its inner penetrable layer (5).

### TECHNICAL FIELD

The invention concerns a device for sealing a natural or artificial intestinal outlet, comprising an inflatable balloon with a roughly toroidal structure, formed of a flat, everted tube segment whose two ends extend roughly coaxially one inside the other and are each connected to a (respective) sleeve, the outer layer of the everted tube segment comprising a radially expanded, patient-proximal region that is to be inserted in the rectum and a patient-distal region, narrowed with respect thereto, that remains outside the rectum during use, and the tube segments having in the transanal region a material hardness H₁, determinable according to the Shore A hardness test, that is greater than 60.

### PRIOR ART

Such tube segments, which are everted and can then be unfolded to a balloon by being inflated, can advantageously be used to seal the rectum or a colostomy. As the balloon is inflated, however, the inner layer of the tube segment is pressed inward, thereby sealing a lumen that is present there. On the other hand, there is subsequently a need to empty the bowel at regular intervals with a minimum of trouble, i.e., if possible without removing and then reinserting the intestinal seal, an operation that is complicated by the sealed inner layer of tubing.

### EXPOSITION OF THE INVENTION

The object underlying the invention is so to improve a device of the aforesaid species for sealing a natural or artificial intestinal outlet that continuous emptying of the bowel is feasible without removing the intestinal seal.

To solve this problem, the invention provides that a stiffening sleeve is inserted in the patient-proximal region of the outer layer in such fashion as to be completely separated from the hollow space inside the balloon by the continuous inner layer thereof.

This sleeve thus keeps the inner lumen in the patient-proximal region of the balloon constantly open and thereby simplifies natural defecation. The transanal region, by contrast, can be closed but also opened, in the manner of a valve.

It is within the scope of the invention that the material hardness H₁, determinable according to the Shore A hardness test, of the tube segments in the transanal region is greater than 70, preferably greater than 80, so that on the one hand, production is possible by extrusion, and on the other hand the expansion that occurs when the balloon is inflated is predictable and limited in extent.

The wall thickness of the balloon, particularly in the region of its outer layer, should be less than 50 μm, preferably less than 40 μm, particularly less than 30 μm. This makes for sufficient pliability, despite the relatively high material hardness H₁, so that this region can be collapsed to a minimal cross section during insertion and between evacuation phases.

The invention recommends using polyurethane for a tube segment forming the balloon. On the one hand, this material possesses the necessary material hardness and can be preshaped to the desired extent; furthermore, it can be fabricated with a very thin wall thickness.

It has proven effective for the stiffening sleeve to have a material hardness H₂ determinable according to the Shore A hardness test that is equal to or less than the material hardness H₁ determinable according to the Shore A hardness test of the tube segments in the transanal region. Since the stiffening sleeve is fashioned as significantly thicker than the balloon and in addition often has to remain in the rectum of a patient along with the balloon as many as 30 days or more, it must not be too hard so as to avoid irritating or even injuring the intestinal mucosa. This is achieved via a limited hardness H₂ for the sleeve material.

One option is to make the tube and the stiffening sleeve out of the same material. The processing of identical materials usually markedly reduces the expenditure involved in machine-based production; in addition, bonding can be done easily and in most cases reliably by slight, temporary dissolution of the two parts that are to be joined together, using a solvent suitable for the material in question. This therefore eliminates the need for costly adhesive bonding preparations.

It has also proven favorable for the material hardness H₂ and the wall thickness d of the stiffening sleeve to be selected such that these elements can be compressed radially for insertion in the rectum. These two parameters affect the actual hardness of the stiffening sleeve, for example in a multiplicative manner. This means that the harder the material of the stiffening sleeve, the thinner-walled the sleeve must be, and vice versa.

The stiffening sleeve should not be connected to either end of the tube segment forming the balloon, so that it can follow every movement of the anterior, patient-proximal portion of the balloon. This gives this balloon segment its great freedom of movement and enables it to roll outward and/or backward in response to the internal pressure generated during inflation, movements that are desirable in this case, as will be explained further hereinbelow.

This inventive idea can be developed further by having one or preferably both ends of the tube segment forming the balloon be disposed in the transanal region(s) or therebeyond (remotely from the patient). Both ends of the tube segment are therefore disposed outside the rectum, so that these regions are readily accessible and, moreover, no sealing problems arise.

A sleeve connected to at least one end of the tube segment forming the balloon is preferably configured as an extracorporeal connector element. Various instruments can then be connected thereto, for example flushing devices, catheters leading to a receptacle remote from the patient, etc.

The fact that the sealed-off volume inside the balloon can be pressurized from the outside makes it possible to deliberately influence its geometrical shape.

According to the invention, a passage surrounded by the balloon and serving to empty the bowel is created by causing the pressurizable volume to be bounded by two roughly mutually concentric surface regions of the balloon. This completely eliminates the need for a shaft or the like, the function of which is completely assumed by the concentric layers of the balloon.

Taking this inventive idea farther, it can additionally be provided that to keep the emptying passage open in the region of the device residing in the anal canal, the inner surface region of the balloon is connected punctiformly, linearly or areally to the outer surface region of the balloon, for example by welding or gluing. In such cases the inner layer of balloon or tubing is affixed to the outer layer.

The invention is further characterized by an occluding balloon for sealing the emptying passage. Such an occluding balloon is fixed in the region of the central lumen of the balloon per se and can be unfolded separately from the balloon per se, for example via a separate feed line through which a preferably gaseous medium is conducted into the occluding balloon.

The occluding balloon is preferably positioned inside the stiffening sleeve, where when deflated it frees up the passage.

The invention further provides that the occluding balloon be formed of a thin-walled, suitably preshaped material. The shape of the occluding balloon when inflated can thus be specified fairly exactly and the pressure needed to unfold the balloon can be kept relatively low, since it need not cause any elastic expansion of the balloon material.

Within an alternative embodiment of the invention, the occluding balloon can be formed by a fully elastically restorable compartment that can be shaped into the balloon. This is possible because the occluding balloon is positioned inside the central lumen of the inventive device and therefore does not come into contact with human tissues, so that even if the internal pressure is too high, no damage can be caused to human tissue.

It is very advantageous to provide in the anterior region of the balloon, particularly in the region of the stiffening sleeve or even patient-proximal thereto, a flushing opening connected to a conduit that extends along the transanal segment and serves to introduce a flushing fluid. In this way, an enema can be given at any time without removing the inventive device.

If the conduit for introducing a flushing fluid extends as far as the anterior, patient-proximal balloon shoulder, then during an enema no bacteria will be flushed out of the transanal region into the bowel, but instead the flushing fluid will pass directly into the bowel without contamination of any kind.

The invention is further optimized by a radial expansion in the transanal segment. This expansion is also intended to serve as a counter-element to the balloon-shaped expansion in the intrarectal tube segment. Its function is to keep the transanal region of the device at least partially outside the anus when a tractive force is developed by the inflation of the balloon, so that the inventive device has a defined position.

For this purpose, the outer layer of the tube segment forming the balloon can be provided in the transanal region with a preshape comprising an outwardly oriented expansion. As a result, a correspondingly preshaped segment of the balloon itself forms the transanal or preanal abutment for the axial force exerted in the direction of the anus by the intrarectal, balloon-shaped expansion.

In an advantageous improvement of the invention, the outwardly directed preshape in the transanal region of the outer layer of the balloon has a ring- or disk-shaped geometry. In this way optimum conditions are established consistently, regardless of the rotational position of the inventive device.

On the other hand, it is also possible for the outwardly oriented preshape in the transanal region of the outer layer of the balloon to include one or two fingers that diverge roughly diametrically from each other. These finger-like extensions can be placed in the anal folds to maximize wearing comfort.

A wedge-shaped element can further be provided, particularly fastened, over the outer layer of the tube segment forming the balloon, in the transanal region or in the region of the transition from the transanal region to the connector element. A foam element of this kind can also serve as an abutment and additionally has increased rigidity, accompanied as a result by very good positional stability.

### BRIEF DESCRIPTION OF THE DRAWING

The invention is explained in greater detail below with reference to some exemplary embodiments.

In the figures:

FIG. 1 shows a first embodiment of the invention in perspective representation;

FIG. 2 is a longitudinal section through FIG. 1 along line II-II; and

FIG. 3 is a partial cutaway view, corresponding to FIG. 2, of a modified embodiment of the invention.

### EXECUTION OF THE INVENTION

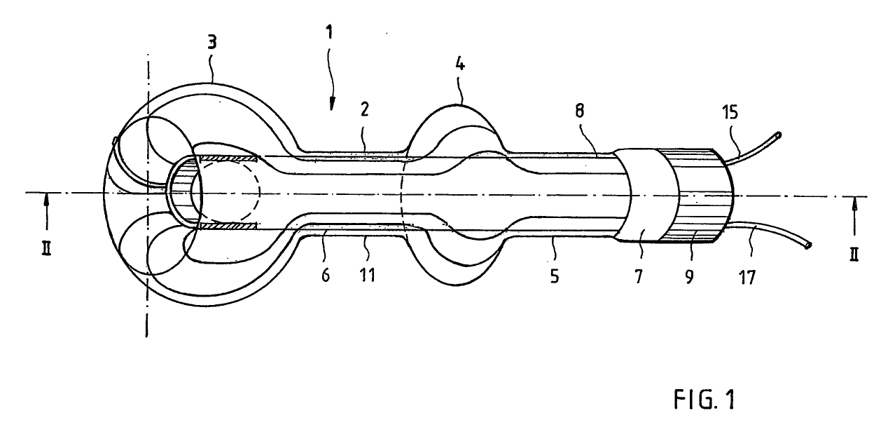

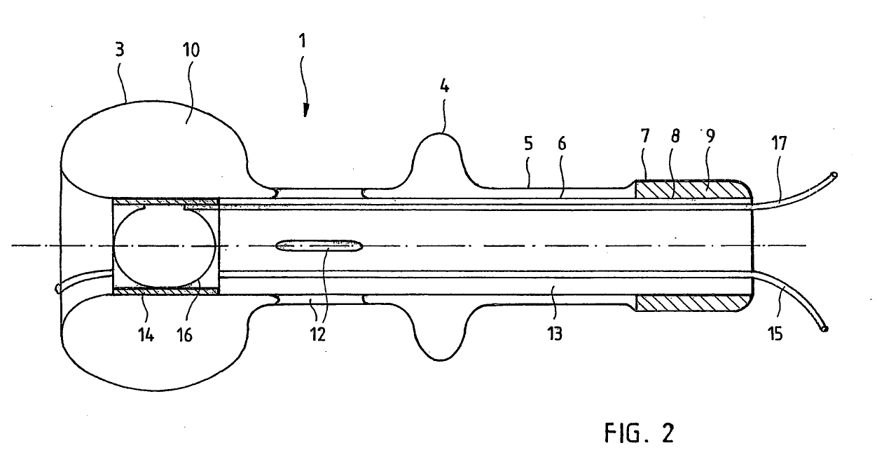

The inventive device 1 according to FIGS. 1 and 2 serves to seal a natural or artificial intestinal outlet. It includes a preshaped and everted tube 2 made of a thin-walled material, for example polyurethane with a Shore A hardness of 90 and a wall thickness of less than 25¹ μm. ¹ Translator's Note: The copy is hard to read, could be 26.

The tube 2, whose original diameter is roughly between 15 and 30 mm, has been provided during the preshaping with two radial expansions 3, 4. The one, preferably larger expansion 3 is disposed roughly in the middle of the tube, which after eversion forms the patient-proximal end of the device 1. The other preshape 4 is located roughly in the middle of the tube segment 5 that is on the outside after eversion, while the internal tube segment 6 is not provided with an expansion, instead having an invariant cross section.

Both free ends 7, 8 of the two tube segments 5, 6 are connected to a sleeve-shaped connector element 9. Connector element 9 can be provided—particularly in the region of its patient-distal end—with an inner and/or outer thread operative to connect various types of medical apparatus, for example a disposal bag, a catheter or the like.

The outer layer 5 of balloon 2 is preferably fixedly glued to the outside of connector element 9, and the inner layer 6 of balloon 2 to the inside thereof. The hollow space 10 between the inner and outer layers 5, 6 of balloon 2 is thereby sealed air-tight; only in the region of connector element 9 is there a connection to the outside (not shown in the drawing), to which a source containing a pressurizable medium can be connected in order to unfold the balloon 2.

Due to its relatively high material hardness, balloon 2 is only very slightly elastic, so that when inflated it assumes the shape, discernible in FIG. 1, prescribed by the preshaping. This includes a roughly cylindrical basic shape comprising a roughly spherical expansion 3 at the patient-proximal end, i.e. the end opposite connector element 9, and comprising a ring- or disk-shaped expansion 4 roughly in the middle between the two ends 3, 9.

The spherical expansion 3 is placed, deflated, in the rectum of a patient (intrarectal segment), while the cylindrical segment 11 adjoining it and extending to the ring- or disk-shaped expansion 4 leads to the outside through the anal canal (transanal region); the patient-distal expansion 4 is located just before the anus, preanally, in the anal fold.

In the transanal region 11 between the two expansions 3, 4, the two layers of the balloon 2, i.e. outer layer 5 and inner layer 6, are connected to each other, preferably by welds 12 or adhesive bonds. These joins can be punctiform, linear or areal welds. The embodiment shown provides four weld lines 12 extending in the axial direction, each offset from the next by roughly equal circumferential angles. By virtue of these weld joints 12, the inner lumen 13 inside the inner layer 6 of the balloon 2 can be opened more easily when the bowel is to be emptied.

Should a contractive movement of the rectal musculature occur during spontaneous emptying of the rectum, the resulting force is absorbed by intrarectal balloon segment 3. The resulting increase in pressure throughout occluding element 1 leads to an active straightening of transanal segment 11 and 5, while the outleading transanal lumen widens and assumes its full cross-sectional area, with a correspondingly large increase in the balloon filling pressure. A further advantage of this movement of pressure and volume from the intrarectal into the transanal segment is the active unwinding or detorquing of the transanal segment. Without such an unwinding mechanism, there is no way of reliably ensuring that the lumen will not become sealed, thereby impairing function, as a result of axial torsion in the transanal segment of the device. The opening of the anal canal, which actively supports the spontaneous defecatory process, can also be adjusted deliberately by the user. For example, should a flushing of the bowel (enema) be performed, the filling pressure in the device 1 can be increased appropriately for the duration of the flushing procedure. A number of functional components cooperate favorably in this case. The axially directed movement of the intrarectal balloon components causes the intrarectal balloon body 3 to conform snugly to the anus that is to be sealed, while the preanal abutment balloon 4 is pulled from the outside against the anus with equal force; in addition, the draining lumen of transanal segment 5 and 11 straightens out and any twists about the longitudinal axis of the device unwind.

In the region of the patient-proximal expansion 3, the inner lumen 13 is held open by a stiffening sleeve 14 whose length is preferably equal to or less than the axial extent of the radial, intrarectal expansion 3 of the balloon 2. The material hardness of the stiffening sleeve 14 is preferably equal to or less than the material hardness of the balloon; the sleeve 14 obtains its stiffness from its increased wall thickness. The same material can preferably be used for the sleeve 14 as for the tube or balloon 2. This makes it easier to fix the sleeve 14 inside the inner lumen 13, particularly by gluing it to the inner layer 6 of the balloon 2, in which case an agent that slightly dissolves the material concerned can be used as glue or for welding.

A tube 15 preferably extends inside inner lumen 13 from the patient-proximal end 3 to the far side of connector element 9. Through this tube 15, which can be affixed, for example by gluing, to the balloon 2—preferably to the inner layer 6 thereof—a flushing medium can be introduced into the bowel of the patient. So that no bacteria can be entrained into the bowel from the transanal region 11 or from the region beyond the transanal expansion 4, the opening of tube 15 is located in the anteriormost region of the intrarectal end 3 of the device 1. Tube 15 can be passed through the inside of stiffening sleeve 14 or between the latter and inner layer 6.

To block central lumen 13 in optimal fashion during an enema, an occluding balloon 16 is also provided. This is preferably disposed inside stiffening sleeve 14, and in the embodiment of FIGS. 1 and 2 has a spherical preshape with a diameter that is slightly greater than the diameter of stiffening sleeve 14. The inflated occluding balloon 16 thereby completely seals central lumen 13 and bears in all circumferential directions, under pressure and slight deformation, against the inside of stiffening sleeve 14, thereby sealing it.

Opening into occluding balloon 16 is an additional tube 17, by which a preferably gaseous pressurizing medium can be conducted into occluding balloon 16 in order to seal lumen 13 in the region of stiffening sleeve 14. Tube 17 is passed through the inside of balloon inner layer 6 and on through connector element 9, and is therefore accessible from the outside. Roughly in the region where tube 17 opens into occluding balloon 16, the latter is affixed to stiffening sleeve 14, for example glued to it in a roughly punctiform manner.

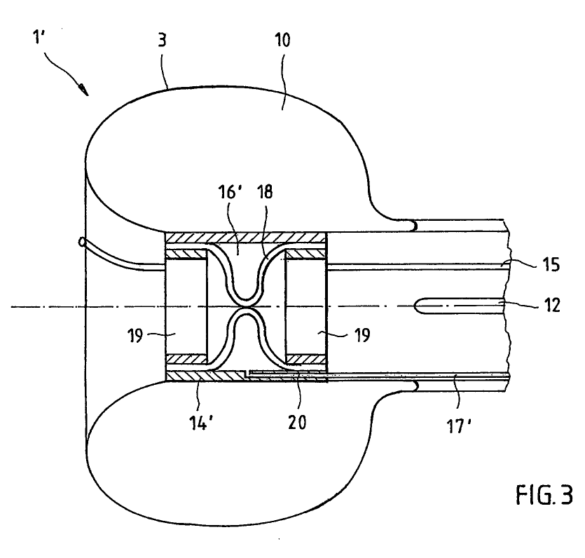

Embodiment 1′ of FIG. 3 differs from the foregoing only in that occluding balloon 16′ has a different configuration. In this last embodiment 1′, this element is configured as short tube 18 having roughly the same diameter and length as stiffening tube 14. This tube 18 can be preshaped in the form of a radial expansion in its axial middle segment. Both ends of this tube 18 are affixed to the inside of stiffening sleeve 14, for example by being clamped between it and a respective inner sleeve 19 inserted in each end. The two inner sleeves 19 can in addition be fixed in stiffening sleeve 14 with glue and then form a structural unit with it. If the hollow space 16′ between the medial, undamped region of tube 18 and stiffening sleeve 14 is filled with a fluid from the outside, then tube 18 buckles inward there, as can be seen in FIG. 3, and seals the lumen 13 in the manner of an iris. For filling with a fluid, in this case stiffening sleeve 14 is provided with a conduit 20 that passes all the way through it from the patient-distal side to the region between the two inner sleeves 19, where it feeds into the inside. Tube 17′ communicates with conduit 20.

1. A device (1, 1′) for sealing a natural or artificial intestinal outlet, the device comprising an inflatable balloon (2) with a generally toroidal structure, formed of a flat, everted balloon segment whose two ends (7, 8) extend generally coaxially one inside the other and are each connected to a respective sleeve (9), the outer layer (5) of the everted tube segment comprising a radially expanded, patient-proximal region comprising an intrarectal region (3) that is to be inserted in the rectum and a patient-distal region (11, 7, 8) comprising a transanal region (4, 11) narrowed with respect thereto, that remains at least regionally outside the rectum during use, and said tube segments having in the transanal region a material hardness H₁, determinable according to the Shore A hardness test, that is greater than 60, wherein a stiffening sleeve (14) is inserted in the intrarectal region (3) of an inner layer (6) in such fashion as to be completely separated from a hollow space (10) inside the balloon (2) by the continuous inner layer (6) thereof.

2. The device as in claim 1, wherein the material hardness H₁, determinable according to the Shore A hardness test, of said tube segments (5, 6) in said transanal region (7, 8, 11) is greater than 70.

3. The device as in claim 1, wherein the wall thickness of said balloon (2), in the region of its outer layer (5), is less 30 μm.

4. The device as in claim 1, wherein the tube segment forming said balloon (2) is made of polyurethane.

5. The device as in claim 1, wherein said stiffening sleeve (14) has a material hardness H₂, determinable according to the Shore A hardness test, that is equal to or less than the material hardness H₁, determinable according to the Shore A hardness test, of said tube segments in said transanal region (7, 8, 11).

6. The device as in claim 1, wherein said balloon (2) and said stiffening sleeve (14) are made of the same material.

7. The device as in claim 1, wherein the hardness and wall thickness of said stiffening sleeve (14) are selected such that the aforesaid elements can be compressed radially for insertion with the rectum.

8. The device as in claim 1, wherein said stiffening sleeve (14) is not connected to either end (7, 8) of the tube segment forming said balloon (2).

9. The device as in claim 1, wherein both ends (7, 8) of the tube segment forming said balloon (2) are disposed in at least one of (i) said transanal regions (4, 11) and (ii) therebeyond remotely from the patient.

10. The device as in claim 1, wherein the sleeve (9) connected to at least one end (7, 8) of the tube segment forming said balloon (2) is configured as an extracorporeal connector element.

11. The device as in claim 1, wherein a sealed-off volume (10) inside said balloon (2) can be pressurized from the outside.

12. The device as in claim 11, wherein a passage (13) surrounded by said balloon (2) and serving to empty the bowel is provided by causing the pressurizable volume (10) to be bounded by two generally mutually concentric surface regions (5, 6) of said balloon (2).

13. The device as in claim 12, wherein to keep the emptying passage (13) open in the region of said device (1; 1′) residing in the anal canal, the inner surface region (6) of said balloon (2) is connected to the outer surface region (5) of said balloon (2) by a selected one of welds (12) and adhesive bonds.

14. The device according to claim 12 and further comprising an occluding balloon (16; 16′) for sealing said emptying passage (13).

15. The device as in claim 14, wherein said occluding balloon (16; 16′) is positioned inside said stiffening sleeve (14).

16. The device as in claim 14, wherein said occluding balloon (16; 16′) is formed from a thin-walled, suitably preshaped material.

17. The device as in claim 14, wherein said occluding balloon (16; 16′) is formed from a fully elastically restorable compartment that can be shaped into said balloon.

18. The device in accordance with claim 1, wherein provided in the region of said stiffening sleeve (14) is a flushing opening connected to a conduit (15) that extends along the transanal segment and serves to introduce a flushing fluid.

19. The device in accordance with claim 18, wherein said conduit (15) for introducing a flushing fluid extends to the anterior, patient-proximal balloon shoulder.

20. The device in accordance with claim 1, and further comprising a radial expansion (4) in said transanal region (7, 8, 11).

21. The device as in claim 20, wherein the outer layer (5) of the tube segment forming said balloon (2) is provided in said transanal region (7, 8, 11) with a preshape (4) comprising an outwardly oriented expansion.

22. The device as in claim 21, wherein the outwardly oriented preshape (4) in said transanal region (7, 8, 11) of said outer layer (5) of said balloon (2) has a ring- or disk-shaped geometry.

23. The device as in claim 21, wherein the outwardly oriented preshape (4) in said transanal region (7, 8, 11) of said outer layer (5) of said balloon (2) is provided with two fingers that diverge diametrically from each other.

24. The device as in claim 20, wherein a wedge-shaped element is provided, particularly fastened, in said transanal region (7, 8, 11) over said outer layer (5) of said tube segment forming said balloon (2).
